(19) United States
(12) Patent Application Publication
(10) Pub. No.: US 2021/0361797 A1
(43) Pub. Date: Nov. 25, 2021

(54) SYSTEMS AND METHODS FOR PATHOGEN PROLIFERATION REDUCTION

(71) Applicants: Paul Binun, Chula Vista, CA (US); Jim Alwan, Chula Vista, CA (US)

(72) Inventors: Paul Binun, Chula Vista, CA (US); Jim Alwan, Encinitas, CA (US)

(21) Appl. No.: 17/326,526

(22) Filed: May 21, 2021

Related U.S. Application Data
(60) Provisional application No. 63/028,935, filed on May 22, 2020.

(51) Int. Cl. A61L 2/10; G02B 6/10; A61L 2/24

(52) U.S. Cl. CPC A61L 2/10; G02B 6/102; A61L 2202/14; A61L 2202/11; A61L 2/24

(57) ABSTRACT

A system for reducing pathogen proliferation on a device includes a waveguide disposed on a body of the device and including a first layer of transparent material having a first refractive index greater than a second refractive index of the body of the device and a third refractive index of an environment in contact with an outer surface of the first layer. A first light source is configured to emit light having a first range of wavelengths into the first layer. The light is substantially confined within the waveguide by total internal reflection. Total internal reflection is frustrated at points of contact between the pathogens or a medium in which the pathogens are suspended and an outer surface of the waveguide, thereby scattering a portion of the light out of the waveguide and into the pathogens and, thereby, reducing proliferation of the pathogens. Related methods are also provided.

### CROSS-REFERENCE TO RELATED APPLICATIONS

This application claims the benefit of the filing date of U.S. Provisional Patent Application No. 63/028,935, filed on May 22, 2020 and entitled “Systems and Methods for Pathogen Proliferation Reduction,” the disclosure of which is hereby incorporated by reference herein in its entirety.

### BACKGROUND

The present disclosure relates to systems for pathogen proliferation reduction and associated methods.

Proliferation of pathogens on surfaces are responsible for a number of serious infections. Of particular interest are the proliferation of pathogens on surfaces of medical equipment in a clinical setting that leads to hospital or clinic-acquired infections and, potentially, eventually to a sepsis and/or death. The worst pathogen strains are those that are resistant to treatment and antibiotics, such as MRSA. Preventing or even restricting pathogen proliferation to a manageable level can save many lives. When the proliferation of pathogens on medical equipment is prevented, the probability of infection is reduced and this frees up resources to combat the more serious types of pathogens.

It is well known that ultra-violet (UV) light is an active mechanism in counteracting bio-pathogens and their proliferation. Many UV sterilization device makers tout their ability to perform tasks anywhere, from disinfecting drinking water to purifying air. However, the use of UV light in preventing bio-pathogen infections can also lead to health problems for humans and animals if the UV light is not contained. Overexposure to UV light can cause direct changes to DNA, which can result in skin cancer. Overexposure to UV light can also act as a photosensitizer and re-activate latent viruses (viral infections that otherwise remain dormant in humans, not producing any symptoms or reductions in quality of life). Unfortunately, these harmful effects limit the usefulness of UV light and where it can be used. For this reason, many UV sterilization devices are sold as chamber disinfectants where the air or liquid to be disinfected must pass through a chamber to be sterilized. While this reduces human overexposure to UV light, it leaves many surfaces unsterilized.

Accordingly, a need exists for systems for pathogen proliferation reduction and associated methods that safely contain sterilizing UV light, where containment is desired, and allow controlled UV light exposure, where pathogen proliferation reduction is desired.

### SUMMARY

In some embodiments, a system for reducing proliferation of pathogens on a device is provided. The system includes a waveguide disposed on a body of the device. The waveguide includes a first layer of transparent material that has a first index of refraction greater than both a second index of refraction of the body of the device and a third index of refraction of an environment in contact with an outer surface of the first layer. The system includes a first light source configured to emit light having a first range of wavelengths into the first layer of transparent material. The light is substantially confined within the waveguide by total internal reflection. The total internal reflection is frustrated at points of contact between the pathogens or a medium in which the pathogens are suspended and an outer surface of the waveguide, thereby scattering a portion of the light out of the waveguide and into the pathogens and, thereby, reducing proliferation of the pathogens.

In some other embodiments, a method of reducing proliferation of pathogens on a device is provided. The method includes launching light having a first range of wavelengths from a first light source into a first layer of transparent material of a waveguide disposed on a body of the device. The first layer of transparent material has a first index of refraction greater than both a second index of refraction of the body of the device and a third index of refraction of an environment in contact with an outer surface of the first layer, thereby substantially confining the light within the waveguide by total internal reflection. The method includes scattering a portion of the light out of the waveguide and into the pathogens at points of contact between the pathogens or a medium in which the pathogens are suspended and an outer surface of the waveguide by frustrating the total internal reflection, thereby reducing proliferation of the pathogens.

In some other embodiments, a method of manufacturing a system for reducing proliferation of pathogens on a device is provided. The method includes providing a substrate having a first refractive index. The method includes disposing a waveguide on the substrate, the waveguide having a second refractive index greater than the first refractive index.

### BRIEF DESCRIPTION OF THE DRAWINGS

A more complete appreciation of the subject matter of the present disclosure and of the various advantages thereof can be realized by reference to the following detailed description in which reference is made to the accompanying drawings in which:

FIG. 1 is a schematic diagram of a system for pathogen proliferation reduction, according to some example embodiments;

FIG. 2 is a side view of at least a portion of a system for pathogen proliferation reduction, according to some example embodiments;

FIG. 3A is a side view of at least a portion of another system for pathogen proliferation reduction, according to some example embodiments;

FIG. 3B is an illustration of a tablet computer with a touch screen protected with an embodiment of the inventions described herein;

FIG. 4 is a side view of at least a portion of yet another system for pathogen proliferation reduction, according to some example embodiments;

FIG. 5 is a flowchart related to a method of pathogen proliferation reduction, according to some example embodiments; and

FIG. 6 is a flowchart related to a method of manufacturing a system for pathogen proliferation reduction, according to some example embodiments.

### DETAILED DESCRIPTION

The present disclosure relates to systems for pathogen proliferation reduction and associated methods. Several inventive embodiments are described below, which utilize UV and/or visible light to disable pathogens according to one of at least the following two mechanisms. First, sufficiently energetic UV light mutates DNA/RNA molecules and prevents their reproduction. Second, UV and at least blue/violet visible light generate toxic reactive oxygen species (ROS) directly within irradiated pathogens that decrease their proliferation. One such class of ROS are hydroxide radicals (OH*), which are oxidizing agents having a neutral charge and a high-affinity for electrons formed when water molecules have a hydrogen atom removed and, so, are ubiquitous with humidity or moisture.

UV light is commonly defined as electromagnetic radiation having a wavelength of between 10-400 nanometers (nm), with wavelengths of 315-400 nm generally corresponding to UV-A light, wavelengths of 280-315 nm generally corresponding to UV-B light, and wavelengths of 100-280 nm generally corresponding to UV-C light. Visible light is defined as electromagnetic radiation having wavelengths in a range as narrow as 420-680 nm, and as broad as 380-800 nm, but is commonly defined as electromagnetic radiation having a wavelength of 400-700 nm, with red, orange, yellow, green, blue and violet light generally being defined as wavelengths between 620-750 nm, 590-620 nm, 570-590 nm, 495-570 nm, 450-495 nm and 380-450 nm, respectively. Accordingly, light used to achieve one or both of the above-described mechanisms of pathogen proliferation reduction can include but is not limited to wavelengths of 300-500 nm for embodiments described herein. Rather, the present disclosure contemplates the utilization of light having any suitable wavelength and/or frequency in the UV and/or visible electromagnetic radiation spectrums.

As will be described in more detail according to one or more embodiments below, devices that experience large tactile traffic can have housings comprising a shell of one or more layers of UV-transparent material or coated with one or more outer films of UV-transparent material that allow light to be launched into, and guided along, the UV-transparent material or coating's surface by total internal reflection (TIR). Many consumer devices already incorporate such UV-transparent materials, including but not limited to medical devices, medical consumables, touch pads in store checkout lines and in personal communication and electronic devices.

In some such embodiments, UV light is propagated in a planar-type membrane waveguide enveloping the surface to be sterilized. Contaminants in contact with the surface scatter the UV light (frustrating the TIR) and propagate UV light out of the waveguide. The low-level UV light propagated out of the waveguide doses the contaminants, thereby neutralizing, sterilizing, and/or deactivating pathogen's ability to reproduce. The dosage of UV light can be selected and/or designed to pass safety standards for light emissions, since most of the light is contained within the waveguide. In some embodiments, a touch or proximity sensor may be deployed to automatically send a signal to turn the light source off or reduce its intensity, thereby making the device safe to use with respect to human exposure hazards. In some embodiments, when the system is not handled, the light source may remain on continuously or pulse to supply the appropriate dose of light for pathogen deactivation.

Further, as will be described in more detail below, some embodiments may employ doped photocatalytic materials that can be activated by visible blue-green, blue, and/or violet light. Without being limited to any particular mechanism of action, activation of photocatalytic materials produces free charge carriers (electrons or holes) by virtue of photon absorption in a semiconductor bandgap of the photocatalytic material. Such holes capture and separate electrons from the negatively charged hydroxide molecules (OH—), producing short-lived, charge-neutral hydroxyl radicals (OH*) that breakdown bonds of any organic material remaining on the surface. These inorganic photocatalytic coatings, sometimes called “self-cleaning” coatings, have been applied to windows of skyscrapers to keep the windows clean.

However, according to at least some embodiments described herein, if the photocatalytic material is tuned with certain dopants, the material's bandgap can be decreased and photon absorption can be shifted towards lower energies (e.g., toward the red end of the visible electromagnetic spectrum). One advantage of such material tuning is that it allows visible light (which does not mutate DNA/RNA and, so, is harmless to life and humans in this respect) to be used for pathogen proliferation reduction. Another is that the tuning provides a narrower bandgap compared to UV and, so, allows more efficient hydroxyl radical formation. While hydroxyl radicals are not harmless to life, their lifetime as radicals is on the order of nanoseconds. Accordingly, pathogens can be deactivated while the light source is active and the light can be deactivated while a touch or determination of sufficient proximity occurs, so no significant dosage is experienced by humans or animals. As with other embodiments described herein, while the light source(s) is/are on, there is little to no UV scatter into the surrounding environment.

As will be described in more detail below, such embodiments can be realized by a waveguide having a thin film of a photocatalytic material applied thereto. The film can have a thickness of as little as a few atomic layers and draws light from the waveguide closer to the surface due to its, typically, high index of refraction. Such embodiments make it possible to clean a surface by applying a water-wetted towel, and then lightly catalyzing the water on the surface utilizing UV and/or visible light to form hydroxyl radicals that clean the surface without harsh chemicals. Several embodiments will now be described in more detail below.

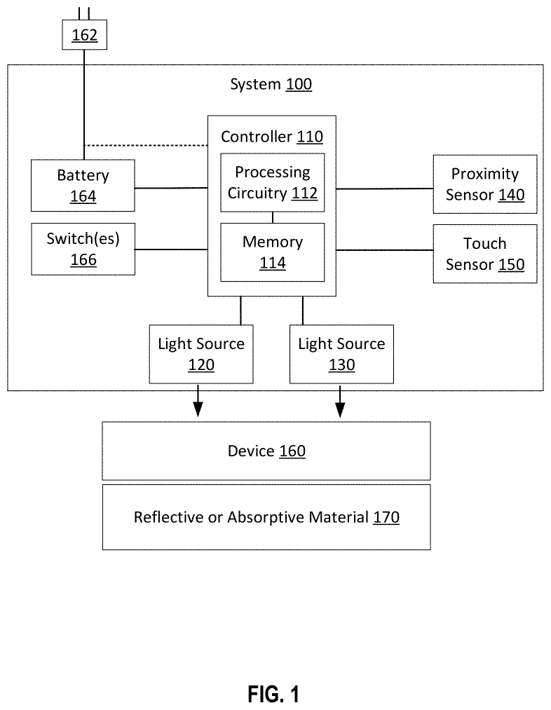

FIG. 1 a schematic diagram of a system 100 for pathogen proliferation reduction, according to some example embodiments. System 100 can include a battery 164 configured to provide power to any component of system 100. Battery 164 can be a disposable and/or rechargeable battery. In some embodiments, system 100 additionally, or alternatively, includes an external power connection 162 configured to provide external power to battery 164 and/or to any powered component of system 100. In some such embodiments external power connection 162 can directly power components of system 100.

System 100 can further include a controller 110 configured to control operation of at least one feature of operation of system 100. In some embodiments, controller 110 can include processing circuitry 112. Processing circuitry 112 can include, but is not limited to, one or more processors, microprocessors, and/or any circuitry suitable for controlling and/or regulating operation of system 100 according to any embodiment described herein. Controller 110 can further include memory 114 configured to store one or more pieces of data for controlling and/or regulating operation of system 100 according to any embodiment described herein. Memory 114 can be a stable data storage, random access memory (RAM), and/or any other suitable type of memory.

System 100 can further include an input device 166, which may be a switch, configured to allow for at least the turning on and off of system 100 (e.g., transitioning system 100 from an ON state to an OFF state, or vice versa). In some embodiments, input device 166 can additionally be configured to receive input from a user that allows for adjustment of one or more aspects of system operation.

System 100 further includes at least a first light source 120 and, in some embodiments, may also or alternatively include at least a second light source 130. Light source 120 can comprise one or more light emitting diodes, laser diodes, fiber lasers, or any other suitable light emitting source configured to emit UV light and arranged in any suitable physical arrangement (e.g., an array). Light source 130 can comprise one or more light emitting diodes, laser diodes, fiber lasers, or any other suitable light emitting source configured to emit light in at least part of the visible spectrum and arranged in any suitable physical arrangement (e.g., an array).

As will be described in more detail below, system 100 can include a front optical surface through which light source(s) 120 and/or 130 are configured to couple with, and emit UV and/or visible light into, at least a portion of a device 160 that may bear a high human handling traffic for pathogen proliferation reduction purposes. Examples of device 160 include, but are not limited to, a tray, door handles, tabletops, a piece of furniture, any surface a passenger may touch on a train, plane or any other form of personal or public transportation, tablet, mobile phone and/or medical device touch panels, hulls of ships and other vessels, waterworks, pools, aquariums, microfluidics and, in general, any water conduit systems. However, system 100 is contemplated for use in any situation in which pathogen proliferation reduction is desired.

In some embodiments, system 100 further includes a reflective or absorptive material 170 that can be disposed on a distal end of device 160. Material 170 is configured to influence the path light emitted from light source(s) 120, 130 ultimately takes through device 160. For example, where material 170 is reflective to at least the wavelengths of light emitted by light source(s) 120, 130, that emitted light may make several passes through a portion of device 160 by virtue of its reflection off of material 170. By contrast, where material 170 is absorptive to at least the wavelengths of light emitted by light source(s) 120, 130, that emitted light may make comparatively fewer passes, or only one pass, through the portion of device 160 by virtue of its absorption upon reaching material 170, for example, at the distal end of device 160.

In some embodiments, system 100 further includes a proximity sensor 140 configured to sense and/or determine if/when an animal or human is within a threshold proximity of system 100. Proximity sensor 140 can comprise circuitry configured to sense when an animal or human is within a threshold proximity of system 100 through any suitable mechanism, for example, capacitive or inductive sensing, motion sensing or radar, lidar or ultrasonic sonar-like sensing. Similarly, in some embodiments, system 100 may further or alternatively include a touch sensor 150 configured to sense and/or determine if/when an animal or human is in physical contact with system 100. Touch sensor 150 can comprise circuitry configured to sense when an animal or human is in physical contact with system 100 through any suitable mechanism, for example, capacitive or inductive touch sensing, or tactile, tremble, motion or acceleration sensing. As will be described in more detail below, one or more of controller 110, proximity sensor 140 and/or touch sensor 150 may be configured to generate a signal that causes a modification to the operation of one or both of light source(s) 120, 130 based on a sensing and/or determining that an animal or human is within a threshold proximity and/or in physical contact with system 100—for example, turning one or both of light source(s) 120, 130 on or off, switch from continuous to pulsed operation or vice versa, and/or switch from a first intensity of emitted light to a second intensity of emitted light different from the first intensity (e.g., either greater or less than the first intensity).

Several embodiments will now be described in connection with system 100 of FIG. 1, with a particular focus on different arrangements of the one or more substantially transparent material layers of, or that are disposed on, device 160 and that are configured to substantially confine the light emitted from light source(s) 120, 130 except at locations of physical contact with, e.g., contaminants, as will be described below.

Example Embodiments Having a Transparent Substrate

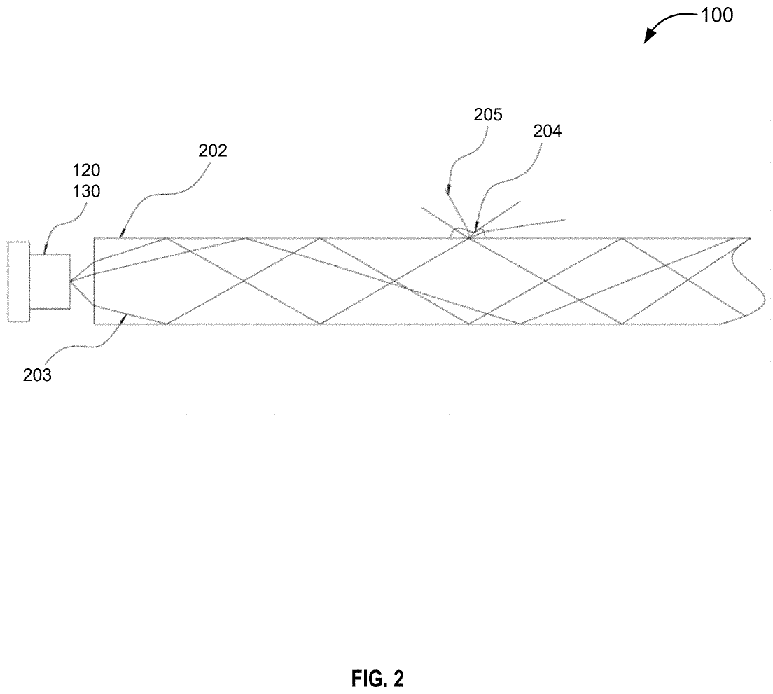

In some embodiments, according to example FIG. 2, light source(s) 120, 130 can be coupled to a bulk, optically transparent volume 202 (e.g., a waveguide) that may bear a high human handling traffic. Waveguide 202 can be disposed on, or be integral to the outer surface of, device 160, for example, a tray, door handles, tabletops, a piece of furniture, any surface a passenger may touch on a train, plane or any other form of personal or public transportation, tablet, mobile phone and/or medical device touch panels. In some embodiments, waveguide 202 may have a thickness within a range of less than 1 micron to a few millimeters. However, the present disclosure is not so limited and any other suitable thickness is also contemplated. In some embodiments, waveguide 202 can comprise a UV-transparent material, for example, a glass such as fused silica, a crystalline material such as sapphire, a ceramic material such as yttria alumina garnet (YAG) and/or aluminum nitride ceramics (AlN), etc., and/or a polymer such as cycloolefin copolymer (COC) and/or cycloolefin polymer (COP). In some embodiments, waveguide 202 can have specular or mildly diffuse surfaces. In some embodiments, such specular or mildly diffuse surfaces can comprise a polished, smooth or slightly matte, but still transparent, surface having a surface roughness within a range of approximately 0.8 to 10 microns.

Waveguide 202 is configured to have an index of refraction (or refractive index) that is higher than the environments immediately adjacent its outside surfaces (e.g., usually air on the outer side and either air, a vacuum, or an outer material of device 160 on the inner side). Accordingly, light 203 launched into waveguide 202 by light source(s) 120, 130 is confined inside the bulk transparent volume of waveguide 202 by total internal reflection (TIF). When a pathogen 204, typically suspended in a medium such as an organic fluid, is deposited onto, for example, an outer surface of waveguide 202, TIF is frustrated at the point of contact with the pathogen 204 (and/or its suspension medium) due to the differing index of refraction between the normal outside environment (e.g., air on an outside of device 160) and the pathogen 204 (and/or its suspension medium) and portions 205 of light 203 are scattered such that they exit into the medium. In this way, light 203 is largely confined within waveguide 202 and only scattered portions 205 of the UV/Violet/Blue light 203 irradiate the organic pathogens 204 suspended in the medium at a constant dosage, thereby reducing or preventing their proliferation.

As previously described in connection with FIG. 1, where a light absorbing material 170 is disposed at the distal end of waveguide 202, light 203 is guided out of the distal end of waveguide 202 and absorbed. By contrast, where a reflective material 170 is disposed at the distal end of waveguide 202, light 203 is reflected back and makes several passes through waveguide 202.

Example Embodiments Having a Waveguide on a Transparent Substrate

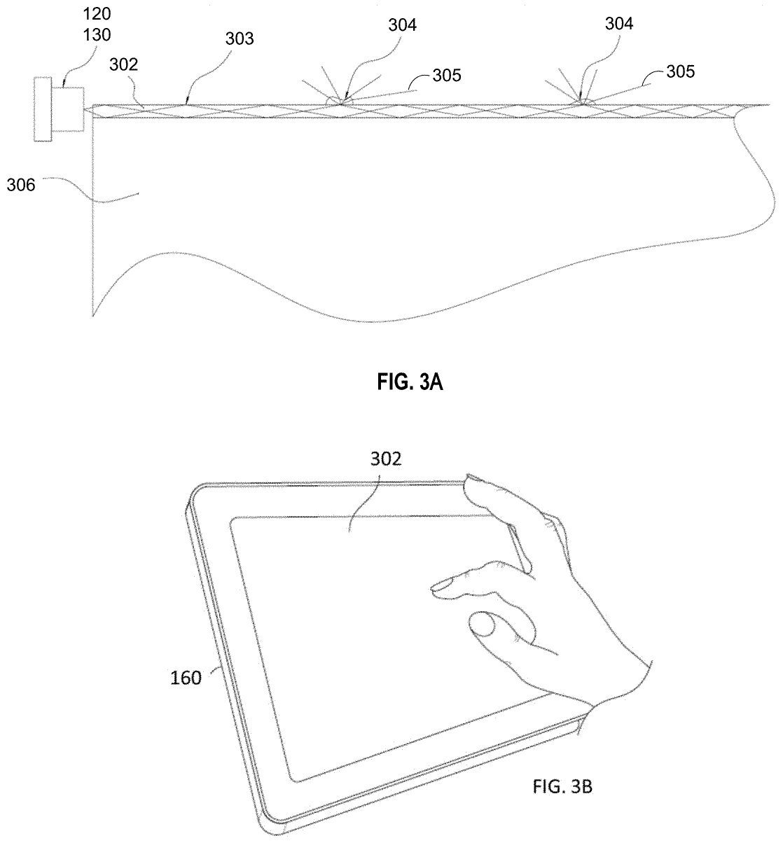

Referring now to FIGS. 3A and 3B, in some embodiments, a body of device 160 can comprise an optically transparent substrate 306 and a waveguide 302 can be disposed in optical contact with substrate 306. In some embodiments, transparent substrate 306 may have a thickness within a range of approximately 10 microns to one centimeter. However, the present disclosure is not so limited and any other suitable thickness is also contemplated. In some embodiments, transparent substrate 306 can comprise a transparent material, for example, a glass such as fused silica, a crystalline material such as sapphire, a ceramic material such as yttria alumina garnet (YAG) or aluminum nitride ceramics (AlN), etc., and/or a polymer such as cycloolefin copolymer (COC) and cycloolefin polymer (COP).

In some embodiments, waveguide 302 is a one- or two-dimensional, planar, round waveguide. However, the present disclosure is not so-limited and also contemplates any other suitable shape for waveguide 302. In some embodiments, waveguide 302 may have a thickness within a range of less than 1 micron to a few millimeters. However, the present disclosure is not so limited and any other suitable thickness is also contemplated. In some embodiments, waveguide 302 can comprise a UV-transparent material, for example, a glass such as fused silica, a crystalline material such as sapphire, a ceramic material such as yttria alumina garnet (YAG) or aluminum nitride ceramics (AlN), etc., and/or a polymer such as cycloolefin copolymer (COC) and cycloolefin polymer (COP).

Waveguide 302 is configured to have an index of refraction that is higher than the environments immediately adjacent its outside surfaces (e.g., usually air on the outer side and substrate 306 on the inner side). Accordingly, light 303 launched into a proximal end of waveguide 302 by light source(s) 120, 130 is guided and confined within the volume of waveguide 302 by total internal reflection (TIF). When a pathogen 304, typically suspended in an organic fluid, is deposited onto, for example, an outer surface of waveguide 302, TIF is frustrated at the point of contact with the pathogen 304 (and/or its organic fluid suspension) due to the differing index of refraction between the normal outside environment (e.g., air on an outside of device 160) and the pathogen 304 (and/or its fluid suspension) and scatters portions 305 of light 303 such that they exit into the fluid suspension. In this way, light 203 is largely confined within waveguide 202 and only scattered portions 205 of the UV/Violet/Blue light 203 irradiate the organic pathogens 204 suspended in the fluid suspension at a constant dosage, thereby reducing or preventing their proliferation.

FIG. 3B illustrates an embodiment of a tablet computer with a touch screen implementing the inventive concepts described herein. In this implementation, the touchscreen device 160 can have a waveguide layer 302 such as described above with reference to FIG. 3 disposed over it to reduce proliferation of pathogens ion the surface of the touchscreen. In some implementations, the waveguide can be provided by an existing protective glass or plastic cover conventionally provided on touchscreen devices. In other embodiments, the system can be added to an existing touchscreen after device manufacture.

Example Embodiments Having a Photocatalytic Film

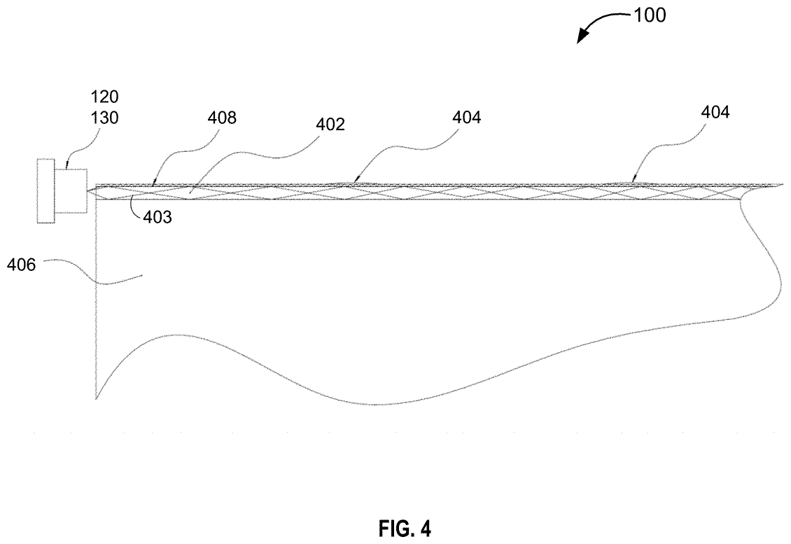

Similar, in part, to embodiments of FIGS. 1 and 3 above, and as illustrated by further reference to FIG. 4, a body of device 160 can comprise an optically transparent substrate 406 and a waveguide 402 can be disposed in optical contact with substrate 406. Waveguide 402 and transparent substrate 406 can correspond substantially to waveguide 302 and substrate 306 of FIG. 3, respectively. An additional photocatalytic film 408 is disposed on top of waveguide 402. In some alternative embodiments, photocatalytic film 408 can be disposed directly on utility substrate 406.

In some embodiments, photocatalytic film 408 may have a thickness within a range of approximately 1 to 100 nanometers. However, the present disclosure is not so limited and any other suitable thickness is also contemplated. In some embodiments, disposing photocatalytic film 408 on, and coupling it to, waveguide 402 may be easier by virtue of photocatalytic film 408 being a relatively thin layer compared to the relatively thicker layer of waveguide 402. In some embodiments, photocatalytic film 408 can comprise TiO₂, Bi₄V₂O₁₁, vanadium pentoxide, and/or any other suitable photocatalytic material.

When light 403 is launched into a proximal end of waveguide 402 and is guided by total internal reflection but escapes into photocatalytic film 408, photocatalytic film 408 itself becomes a part of the waveguide. When light 403 enters photocatalytic film 408, it is absorbed and generates charge “holes” on the film's surface, which strip electrons from hydroxide molecules in existing surface moisture and produce hydroxyl radicals without light 403 exiting the waveguide (e.g., waveguide 402 and photocatalytic film 408). These ROS react with and dissolve contaminant 404, producing a thin film of water on the surface due to the hydrophilic nature of the irradiated surface. As contaminant 404 is dissolved, destruction of internal organics accelerates as more internal reaction occurs. In severely dry weather, utilizing a wet wipe or water-moistened towel may be sufficient to provide an abundance of hydroxide molecules on the device surface.

One advantage of embodiments according to FIG. 4, for example having a photocatalytic film 408 disposed on a waveguide 402 as described, is that more light from light source(s) 120, 130 is drawn to the surface of photocatalytic film 408 and/or of waveguide 402 and, therefore, a higher intensity of UV and/or visible light can be provided at that/those surface(s), requiring less overall power to light source(s) 120, 130 compared to embodiments according to FIGS. 2-3 as described above.

Example Embodiments Having Diffuse Surfaces for Controlled Scatter

Some embodiments may encompass all or a portion of all each of the prior-discussed embodiments. In some such embodiments, surfaces of the waveguide are made deliberately diffuse in order to scatter the light out of the waveguide and irradiate the surfaces in a controlled fashion. For example, additional dopants, e.g., transition metals including but not limited to Chromium (Cr) and Vanadium (V), can be added to any one or more of optically transparent volume 202 of FIG. 2, optically transparent substrate 306 and/or waveguide 302 of FIG. 3, or optically transparent substrate 406, waveguide 402, and/or photocatalytic film 408 of FIG. 4, to induce a deliberate and controlled scatter of light. In some embodiments, the additional dopants may be introduced in predetermined patterns, concentrations, e.g., between approximately 10⁷ and 10⁵ mol/g though any other suitable concentration or range of concentrations are also contemplated, and/or locations of the one or more above-described layers in order to deliberately control selectivity of the wavelengths that are scattered such that, for example, shorter wavelengths of the light sourced (e.g., light having a first wavelength or range of wavelengths) scatter out of the waveguide but longer wavelengths of the light sourced (e.g., light having a second wavelength or range of wavelengths longer than the first) do not, or vice-versa.

Example Operational Schemes for Light Sources

The disclosure now turns to example operational schemes for a light source as described in this disclosure, e.g., light source(s) 120, 130. Light sources 120, 130 can emit light comprising several wavelengths that simultaneously inhibit reproduction and/or kill the pathogens in different ways to speed up the process or increase the efficacy across a broader spectrum of pathogens. A first wavelength of light, in the UV region, is broadly harmful to lifeforms. A second wavelength of light, in the visible, blue-violet region, is not directly harmful to lifeforms.

As described above, emission of this first wavelength of UV light, for example by light source 120, can be automatically controlled by one or more of proximity sensor 140, touch sensor 150 and/or controller 110 (e.g., one or more of these components can be configured to turn off or modify the operation of light source 120 when a proximity to or touch of device 160 sensed, detected and/or determined). Accordingly, the surface of device 160 is only affected by light emission from light source 120 during relatively longer periods without human interaction. And when in operation, the UV light emitted by light source 120 is contained in the waveguide(s) and only doses surface contaminants, as described above. This provides a quicker action affecting a broader spectrum of pathogens by mutating and inhibiting the proper replication of its RNA/DNA. By contrast, and in some same or other embodiments, controller 110 can be configured to cause light source 130 to emit the second, longer blue-violet wavelength light at a constant low dose to prevent the proliferation of pathogens through ROS production, which makes the environment less conducive to starting a colony.

Example Method(s) of Use

The disclosure now turns to one or more example methods of utilizing a system for pathogen proliferation reduction as described anywhere in this disclosure.

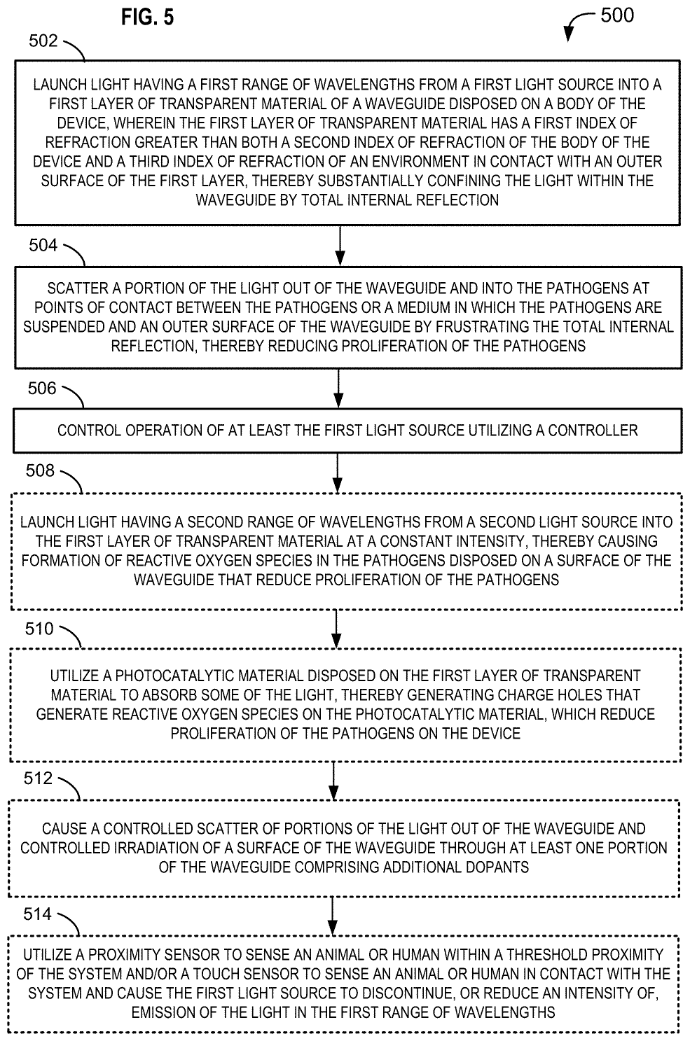

FIG. 5 illustrates a flowchart 500 for an example method of utilizing a system for pathogen proliferation reduction, as described anywhere in this disclosure. Although particular steps are described herein, the present application is not so limited and alternative methods of manufacturing a system for pathogen proliferation reduction may include a subset of these steps, in the same or different order, and may additionally include one or more addition steps not described herein.

Step 502 includes launching light having a first range of wavelengths from a first light source into a first layer of transparent material of a waveguide disposed on a body of the device. The first layer of transparent material has a first index of refraction greater than both a second index of refraction of the body of the device and a third index of refraction of an environment in contact with an outer surface of the first layer, thereby substantially confining the light within the waveguide by total internal reflection. For example, as previously described in connection with at least FIGS. 1-4, light having a first range of wavelengths (e.g., UV light) can be launched from first light source 120 into a first layer of transparent material 202, 302, 402 of a waveguide disposed on a body of the device. The first layer of transparent material 202, 302, 402 has a first index of refraction greater than both a second index of refraction of the body of the device (e.g., not shown in FIG. 2 but shown as substrate 306 in FIG. 3 or substrate 406 in FIG. 4) and a third index of refraction of an environment in contact with an outer surface of the first layer (e.g., air in FIG. 2 or 3, photocatalytic film 408 in FIG. 4), thereby substantially confining the light 203, 303, 403 within the waveguide by total internal reflection.

Step 504 includes scattering a portion of the light out of the waveguide and into the pathogens at points of contact between the pathogens or a medium in which the pathogens are suspended and an outer surface of the waveguide by frustrating the total internal reflection, thereby reducing proliferation of the pathogens. For example, as previously described in connection with at least FIGS. 1-4, a portion 205, 305 of the light 203, 303 is scattered out of the waveguide and into the pathogens 204, 304 at points of contact between the pathogens (and/or the medium they are suspended in) an outer surface of the waveguide by frustrating the total internal reflection, thereby reducing proliferation of the pathogens.

In some embodiments, step 506 includes controlling operation of at least the first light source utilizing a controller. For example, as previously described in connection with at least FIGS. 1-4, controller 110 can control operation of at least first light source 120 as described anywhere in this disclosure.

In some embodiments, optional step 508 includes launching light having a second range of wavelengths from a second light source into the first layer of transparent material at a constant intensity, thereby causing formation of reactive oxygen species in the pathogens disposed on a surface of the waveguide that reduce proliferation of the pathogens. For example, as previously described in connection with at least FIGS. 1-4, light having a second range of wavelengths (e.g., visible light) from second light source 130 into a first layer of transparent material 202, 302, 402 of a waveguide disposed on a body of device 160 at a constant intensity, thereby causing formation of reactive oxygen species in the pathogens disposed on a surface of the waveguide that reduce proliferation of the pathogens.

In some embodiments, optional step 510 includes utilizing a photocatalytic material disposed on the first layer of transparent material to absorb some of the light, thereby generating charge holes that generate reactive oxygen species on the photocatalytic material, which reduce proliferation of the pathogens on the device. For example, as previously described in connection with at least FIGS. 1-4, photocatalytic material 408 disposed on first layer of transparent material 406 is configured to absorb some of the light 403, thereby generating charge holes that generate reactive oxygen species on photocatalytic material 408, which reduce proliferation of the pathogens on device 160.

In some embodiments, optional step 512 includes causing a controlled scatter of portions of the light out of the waveguide and controlled irradiation of a surface of the waveguide through at least one portion of the waveguide comprising additional dopants. For example, as previously described in connection with at least FIGS. 1-4, at least one portion of the waveguide can comprise additional dopants that cause a controlled scatter of portions of the light 203, 303, 403 out of the waveguide and controlled irradiation of a surface of the waveguide.

In some embodiments, optional step 514 includes utilizing at least one of a proximity sensor to sense an animal or human within a threshold proximity of the system or a touch sensor to sense an animal or human in contact with the system and cause the first light source to discontinue, or reduce an intensity of, emission of the light in the first range of wavelengths. For example, as previously described in connection with at least FIGS. 1-4, at least one of proximity sensor 140 to sense an animal or human within a threshold proximity of system 100 or a touch sensor 150 to sense an animal or human in contact with system 100 and cause first light source 120 to discontinue, or reduce an intensity of, emission of the light in the first range of wavelengths.

Example Methods of Manufacture

The disclosure now turns to one or more example methods of manufacturing at least a portion of a system for pathogen proliferation reduction as described anywhere in this disclosure.

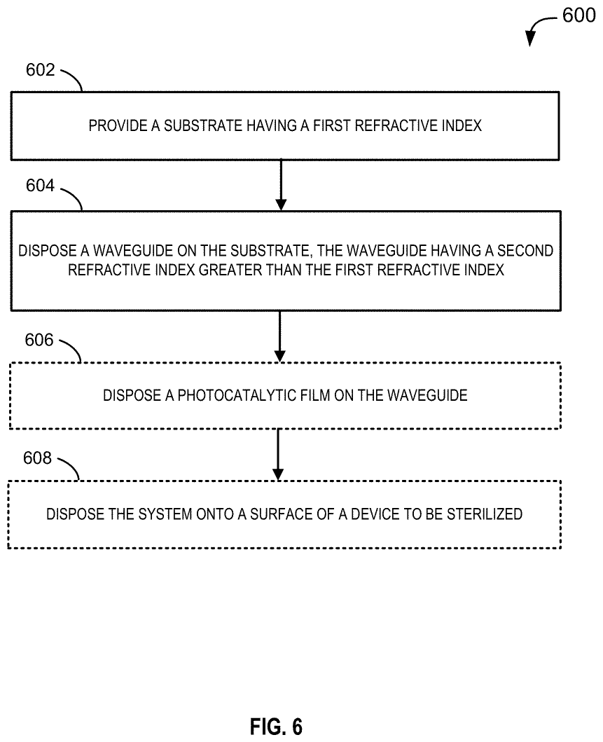

FIG. 6 illustrates a flowchart 600 for an example method of manufacturing a system for pathogen proliferation reduction, as described anywhere in this disclosure. Although particular steps are described herein, the present application is not so limited and alternative methods of manufacturing a system for pathogen proliferation reduction may include a subset of these steps, in the same or different order, and may additionally include one or more addition steps not described herein.

Step 602 includes providing a substrate having a first refractive index. For example, as previously described in connection with at least FIGS. 3 and 4, optically transparent substrate 306 or 406 can be provided. As previously described in connection with at least FIG. 2, such a substrate may comprise an outer layer of device 160.

Step 604 includes disposing a waveguide on the substrate. The waveguide has a second refractive index greater than the first refractive index. For example, with respect to FIG. 2, waveguide 202 can be disposed on a substrate that comprises an outer surface of device 160; with respect to FIG. 3, waveguide 302 can be disposed on optically transparent substrate 306; and with respect to FIG. 4, waveguide 402 can be disposed on optically transparent substrate 406. In some embodiments, waveguide 202, 302, 402 can comprise a sheet of a transparent polymer. However, the present disclosure is not so limited, and contemplates the utilization of any other suitable material that is sufficiently transparent to UV and/or visible light. In some embodiments, waveguide 302, 402 is laminated onto respective optically transparent substrate 306, 406. However, the present disclosure is not so limited, and contemplates any other suitable method of fixing waveguide 304, 402 on respective optically transparent substrate 306, 406.

Optionally, step 606 includes disposing a photocatalytic film on the waveguide. For example, as previously described in connection with at least FIG. 4 above, photocatalytic film 408 can be disposed on at least an outer surface of waveguide 302, 402. In some embodiments, waveguide 402 and substrate 406 can be coated with photocatalytic film 408 using any suitable process.

In some embodiments, such a method of manufacture can further include doping photocatalytic film 408 with dopants that decrease a bandgap of photocatalytic film 408, thereby red-shifting photon absorption of photocatalytic film 408 from the UV range toward the visible range of light.

In some embodiments, such a method of manufacture can further include doping at least a portion of waveguide, e.g., any of layers 202, 302, 402, 306, 406, 408 with dopants sufficient to cause a controlled scatter of a portion of light launched into the waveguide out of the waveguide and, thereby provide controlled irradiation of a surface of the waveguide. In some such embodiments, the dopants cause selective scattering of a selected range of wavelengths of light out of the waveguide.

Optionally, step 608 includes disposing the system onto a surface of a device to be sterilized. In some embodiments, such a surface can include a display of the device. Any suitable adhesive method(s) are contemplated for permanently or removably fixing the structure onto the surface of the device. In some embodiments, light source 120, 130 of system 100 itself, powered by battery 164 or electrical plug 162, is configured to launch the UV and/or visible light into the waveguide structure, as described anywhere herein. In some other embodiments, a display of device 160 itself, on which the laminated structure is disposed, acts as the light source that is configured to launch the UV and/or visible light into the waveguide structure.

In some embodiments, a method related to flowchart 600 can further include providing first light source 120 configured to emit light 203, 303, 403 having a first range of wavelengths into the waveguide.

In some embodiments, a method related to flowchart 600 can further include providing proximity sensor 140 configured to sense an animal or human within a threshold proximity of system 100 and generate a signal that causes first light source 120 to discontinue, or reduce an intensity of, emission of light 203, 303, 403 in the first range of wavelengths. Additionally, or alternatively, a method related to flowchart 600 can further include providing touch sensor 150 configured to sense an animal or human in contact with system 100 and generate a signal that causes first light source 120 to discontinue, or reduce an intensity of, emission of light 203, 303, 403 in the first range of wavelengths.

In some embodiments, a method related to flowchart 600 can further include providing reflective material 170 configured for placement on a distal end of device 160 to, thereby, reflect at least some of light 203, 303, 403 launched into the waveguide back into device 160.

In some embodiments, a method related to flowchart 600 can further include providing absorptive material 170 configured for placement on a distal end of device 160 to, thereby, prevent reflection of at least some of light 203, 303, 403 launched into the waveguide back into device 160.

In some embodiments, a method related to flowchart 600 can further include one or more of: (1) providing second light source 130 configured to launch light having a second range of wavelengths into the waveguide at a constant intensity, thereby causing formation of reactive oxygen species in pathogens 204, 304 disposed on a surface of the waveguide that reduce proliferation of pathogens 204, 304, (2) providing battery 164 configured to power one or more components of system 100, (3) providing electric plug 162 configured to draw power from an external source to power one or more components of system 100, and/or (4) providing a switch 166 configured to transition system 100 between an on state and an off state.

Although the disclosure herein has been described with reference to particular embodiments, it is to be understood that these embodiments are merely illustrative of the principles and applications of the present disclosure. It is therefore to be understood that numerous modifications may be made to the illustrative embodiments and that other arrangements may be devised without departing from the spirit and scope of the present disclosure as defined by the appended claims.

1. A system for reducing proliferation of pathogens on an article subject to pathogen exposure, the system comprising:
a waveguide disposed on a body of the article, the waveguide comprising a first layer of transparent material;
a first light source configured to emit light having a first range of wavelengths into the first layer of transparent material, wherein:
the light is substantially confined within the waveguide by total internal reflection; and
the total internal reflection is frustrated at points of contact between the pathogens or a medium in which the pathogens are suspended and an outer surface of the waveguide, thereby scattering a portion of the light out of the waveguide and into the pathogens and, thereby, reducing proliferation of the pathogens.

2. The system of claim 1, further comprising a controller configured to control operation of at least the first light source.

3. The system of claim 1, wherein the first range of wavelengths at least partially overlap the ultra-violet range of light.

4. The system of claim 2, comprising a second light source configured to emit light having a second range of wavelengths into the first layer of transparent material, the controller further configured to cause the second light source to emit the light in the second range of wavelengths at a constant intensity and, thereby, cause formation of reactive oxygen species in a pathogen disposed on a surface of the waveguide that reduces proliferation of the pathogen.

5. The system of claim 4, wherein the second range of wavelengths at least partially overlap the visible blue-violet range(s) of light.

6. The system of claim 4, wherein the first and second light sources each comprises one or more light emitting diodes, laser diodes, and/or fiber lasers.

7. The system of claim 1, wherein the body of the device comprises an optically transparent substrate in optical contact with the waveguide.

8. The system of claim 1, wherein the waveguide further comprises a photocatalytic material disposed on the first layer of transparent material, the photocatalytic material configured to absorb some of the light, thereby generating charge holes that generate reactive oxygen species on the photocatalytic material, which reduce proliferation of the pathogens on the device.

9. The system of claim 8, wherein the photocatalytic material comprises dopants that decrease a bandgap of the photocatalytic material, thereby red-shifting photon absorption of the photocatalytic material from the UV range toward the visible range of light.

10. The system of claim 1, wherein at least one portion of the waveguide comprises additional dopants that cause a controlled scatter of portions of the light out of the waveguide and controlled irradiation of a surface of the waveguide.

11. The system of claim 10, wherein the additional dopants cause selective scattering of a selected range of wavelengths of light.

12. The system of claim 1, further comprising a reflective material configured to be disposed on a distal end of the device and reflect at least some of the light emitted by the first light source back into the device.

13. The system of claim 1, further comprising an absorptive material configured to be disposed on a distal end of the device and absorb at least some of the light emitted by the first light source such that the absorbed light does not reflect back into the device.

14. The system of claim 2, further comprising a proximity sensor configured to sense an animal or human within a threshold proximity of the system, wherein sensing the animal or human within the threshold proximity causes at least one of the proximity sensor and the controller to cause the first light source to discontinue, or reduce an intensity of, emission of the light in the first range of wavelengths.

15. The system of claim 2, further comprising a touch sensor configured to sense an animal or human in contact with the system, wherein sensing the animal or human in contact with the system causes at least one of the touch sensor and the controller to cause the first light source to discontinue, or reduce an intensity of, emission of the light in the first range of wavelengths.

16. The system of claim 1, further comprising a battery configured to power one or more components of the system

17. The system of claim 1, further comprising an electric plug configured to draw power from an external source to power one or more components of the system.

18. The system of claim 1, comprising a switch configured to transition the system between an on state and an off state.

19. The system of claim 1, wherein the waveguide is planar.

20. The system of claim 1, wherein the device comprises at least one of a tray, a door handle, a tabletop, a piece of furniture, a tablet, a mobile phone, a medical device, a hull of a ship, a pool, an aquarium, waterworks, microfluidics, and a water conduit system.

21-59. (canceled)
